(19) United States
(12) Patent Application Publication
(10) Pub. No.: US 2025/0281368 A1
(43) Pub. Date: Sep. 11, 2025

(54) HAIR CONDITIONING COMPOSITION

(71) Applicant: The Procter & Gamble Company, Cincinnati, OH (US)

(72) Inventor: Nobuaki UEHARA MATSUOKA, Lakeholmz (SG)

(21) Appl. No.: 19/220,397

(22) Filed: May 28, 2025

Related U.S. Application Data
(63) Continuation of application No. 17/350,069, filed on Jun. 17, 2021.
(60) Provisional application No. 63/045,428, filed on Jun. 29, 2020.

(51) Int. Cl. A61K 8/04; A61K 8/02; A61K 8/06; A61K 8/34; A61K 8/35; A61K 8/36; A61K 8/37; A61K 8/44; A61K 8/49; A61Q 5/12; A61Q 17/00

(52) U.S. Cl. CPC A61K 8/042; A61K 8/0233; A61K 8/06; A61K 8/068; A61K 8/342; A61K 8/345; A61K 8/35; A61K 8/361; A61K 8/375; A61K 8/44; A61K 8/4946; A61Q 5/12; A61Q 17/005; A61K 2800/21; A61K 2800/30; A61K 2800/33; A61K 2800/34; A61K 2800/412; A61K 2800/805

(57) ABSTRACT

A hair conditioner composition comprising: a) a basic amino acid; b) a fatty acid; c) a high melting point fatty alcohol; and d) an aqueous carrier; wherein a) through d) form a lamellar gel network matrix with a L_{β} phase.

### FIELD OF THE INVENTION

The present invention relates to a hair conditioning composition comprising a basic amino acid, a fatty acid, a high melting point fatty alcohol, and aqueous carrier, all of which form a lamellar gel network matrix with a L_{β} phase in formulation.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds and an aqueous carrier, are believed to provide a lamellar gel network matrix with a L_{β} phase, which is suitable for providing a variety of conditioning benefits such as a slippery feel during the application to wet hair, softness, and a moisturized feel on dry hair.

But some consumers would prefer products that do not have cationic surfactant molecules. Thus, there is a continuing need to find alternative surfactants, particularly green chemistry surfactants that can still form a lamellar gel network matrix with L_{β} phase in formulation and deliver the consumer-desired benefits.

None of the existing art provides all of the advantages and benefits of the present invention, including performance, cost, safety, sustainable sourcing, and being environmental-friendly.

### SUMMARY OF THE INVENTION

The present invention is directed to a hair conditioning composition comprising a basic amino acid, a fatty acid, a high melting point fatty alcohol, and an aqueous carrier, all of which form a lamellar gel network matrix with a L_{β} phase in formulation.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Q.S. herein means up to 100%.

### Composition

The hair conditioning composition of the present invention comprising:
a) a basic amino-acid;
b) a fatty acid;
c) a high melting point fatty alcohol; and
d) an aqueous carrier;
   wherein a) through d) form a lamellar gel network matrix with a L_{β} phase.

The objective of the invention is to provide stable conditioner compositions containing a lamellar gel network matrix with a L_{β} phase and having a thick yet melting application feel, clean rinse, and dry look and conditioning benefits. The combination of a fatty alcohol, a fatty acid, and a basic amino acid with an aqueous carrier enables the preparation of lamellar sheet structures that allow a good rheology profile to provide good phase stability and wet feel. The lamellar sheet structure is robust enough to provide compositional stability, but it is also sensitive to dilution and shear during the product use to be able to break down and provide excellent wet feel. The lamellar structure and the composition further deliver dry conditioning benefits such as hair manageability, frizz control, and volume.

A typical hair conditioner composition comprises a structurant, such as a fatty alcohol, a feel/rheology modifier polymer, a conditioning oil and agent, preservation ingredients, perfumes/colorants, plus a cationic surfactant. As consumers become more interested in products comprising only natural and gentle ingredients, each of these hair conditioner components is examined for how consumer-perceived natural and gentle ingredients can make up the composition, while still providing the performance and benefits that consumers expect. One area that may be modified is the surfactant. Rather than use a cationic surfactant molecule, the present inventors have formulated hair conditioner compositions comprising a fatty acid, a high melting point fatty alcohol, and a basic amino acid. Integrating different basic amino acids and fatty acids during formulation at certain pH and processing conditions allows flexibility in molecular design and the supply chain, all with natural and gentle ingredients, while still meeting performance, cost, safety, sustainable sourcing and environmental-friendly criteria.

Lamellar Gel Network Matrix with a L_{B} Phase

The compositions of the present invention comprise a lamellar gel network matrix with a L_{β} phase. The lamellar gel network matrix with a L_{β} phase, sometimes referred to as a gel network or gel matrix, comprises the fatty acid, fatty alcohol, basic amino acid, and an aqueous carrier. The lamellar gel network matrix with a L_{β} phase is suitable for providing various conditioning benefits, such as slippery feel during the application to wet hair and softness and moisturized feel on dry hair.

The lamellar gel network matrix with a L_{β} phase of the inventive hair conditioner compositions comprises a surfactant that comprises a basic amino acid and longer alkyl chain of a fatty acid that contain C10-C22 as major chain length.

The composition may comprise from about 0.01% to about 15% of the basic amino-acid, by weight of the hair conditioner composition. In some embodiments, the amount of the basic amino acid may be from about 0.01% to about 15%, preferably from about 0.03% to about 10%, more preferably from about 0.1% to about 6%, by weight of the hair conditioner composition. In some embodiments, the basic amino acid comprises more than two amine groups. Suitable basic amino acids may include, but are not limited to, arginine, lysine, histidine, poly-amino acids, and combinations thereof. In some embodiments, additional amino acids may be added, including poly-amino acids. Poly-amino acids may be added up to a 1:1 ratio with other amino acids. The iso-electrostatic point for each amino acid, and for all amino acids when combined may be at least about 7. In some embodiments, the composition may further comprise di-amines that are not basic amino acids, such as hydroxyethyl urea.

The basic amino acids of the present invention may be combined with a fatty acid at certain pH and processing conditions to form a lamellar gel network matrix with a L_{β} phase. The composition may comprise from about 0.01% to about 15% of the fatty acid, by weight of the hair conditioner composition. In some embodiments, the amount of the fatty acid may be from about 0.01% to about 15%, preferably from about 0.05% to about 10%, more preferably from about 0.1% to about 5%, by weight of the hair conditioner composition. The fatty acid may comprise saturated and/or unsaturated fatty acids. The ratio of saturated fatty acids to unsaturated fatty acids may be from about 8:1 to about 1:4, or from about 4:1 to about 1:2. In some embodiments, the fatty acid may comprise from about 0.3% to about 5% of unsaturated fatty acid, by weight of the hair conditioner composition. The fatty acid may have C10-C22 alkyl chains, in some cases C16-C22 alkyl chains, and in still other cases C18-C22 alkyl chains as the main components. The saturated fatty acid may include, but is not limited to, stearic acid palmitic acid, behenic acid, and combinations thereof. The unsaturated fatty acid may include, but is not limited to, rapeseed acid, oleic acid, linoleic acid, and combinations thereof.

In some embodiments, use of fatty acids having C12-C14 alkyl chains may improve the wet detangling of the hair conditioning composition, while maintaining a clean feel.

In some embodiments, compositions comprising C12-C14 shorter fatty acid alkyl chains can provide improved wet and dry conditioning, in combination with high levels of arginine.

However, in some embodiments, compositions comprising longer fatty acid chains, such as C22, may provide a coated dry conditioning feel without a greasy residue feel, especially for highly damaged hair.

In some embodiments, the surfactant may further comprise cationic surfactants in addition to a basic amino acid. Suitable cationic surfactants may include, for example, behentrimonium methosulfate (BTMS), behentrimonium chloride (BTMAC), stearamidopropyldimethylamine (SAPDMA), behenamidopropyldimethylamine (BAPDMA), brassicyl valinate esylate, and combinations thereof.

The hair conditioner compositions may comprise at least about 60% of an aqueous carrier, by weight of said hair conditioner composition, and in some embodiments at least about 80%.

The lamellar gel network matrix with a L_{β} phase comprises a fatty acid, high melting point fatty alcohol, basic amino acid, and an aqueous carrier. In general, the mixture of the basic amino acid and the fatty acid, along with an aqueous carrier, may have a pH of at least about 4.5. The ratio of basic amino acid to the fatty acid may be from about 1:40 to about 40:1, preferably from about 1:15 to about 30:1, or more preferably from about 1:10 to about 20:1.

In some embodiments, the ratio of the sum of the basic amino acid (a) plus fatty acid (b) to the sum of the basic amino acid (a), fatty acid (b), and fatty alcohol (c), by weight ((a+b)/(a+b+c)), may be from about 0.1 to about 0.9, preferably from about 0.2 to about 0.5. In some embodiments, the conditioner composition may comprise from about 6% to about 20%, by weight of the composition, of the basic amino acid plus fatty acid plus fatty alcohol. These ratios and weight percents may provide better detangling and also a more robust lamellar gel network matrix with a L_{β} phase structure.

The compositions of the present invention may be substantively free of ceramide. The compositions of the present invention may be substantively free of cholesterol. And the compositions of the present invention may be substantively free of a gel network made of only non-ionic surfactant.

### High Melting Fatty Alcohol

The high melting point fatty alcohol can be included in the composition at a level of from about 2%, preferably from about 4%, more preferably from about 5%, still more preferably from about 5.5%, and to about 15%, preferably to about 10% by weight of the composition, in view of providing the benefits of the present invention.

The high melting point fatty alcohol useful herein have a melting point of 25° C. or higher, preferably 40° C. or higher, more preferably 45° C. or higher, still more preferably 50° C. or higher, in view of stability of the lamellar gel network matrix with Lβ. Preferably, such melting point is up to about 90° C., more preferably up to about 80° C., still more preferably up to about 70° C., even more preferably up to about 65° C., in view of easier manufacturing and easier emulsification. In the present invention, the high melting point fatty alcohol can be used as a single alcohol or as a blend or mixture of at least two high melting point fatty alcohols. When used as such blend or mixture, the above melting point means the melting point of the blend or mixture.

The high melting point fatty alcohol useful herein is selected from the group consisting of fatty alcohols, fatty alcohol derivatives, fatty acid derivatives, and mixtures thereof. It is understood by the artisan that the alcohols disclosed in this section of the specification can in some instances fall into more than one classification, e.g., some fatty alcohol derivatives can also be classified as fatty acid derivatives. However, a given classification is not intended to be a limitation on that particular alcohol but is done so for convenience of classification and nomenclature. Further, it is understood by the artisan that, depending on the number and position of double bonds, and length and position of the branches, certain alcohols having certain required carbon atoms may have a melting point of less than the above preferred in the present invention. Such alcohols of low melting point are not intended to be included in this section. Nonlimiting examples of the high melting point alcohols are found in International Cosmetic Ingredient Dictionary, Fifth Edition, 1993, and CTFA Cosmetic Ingredient Handbook, Second Edition, 1992.

The high melting point fatty alcohols useful herein are those having from about 14 to about 30 carbon atoms, preferably from about 16 to about 22 carbon atoms. These fatty alcohols are saturated and can be straight or branched chain alcohols.

Preferred fatty alcohols include, for example, cetyl alcohol (having a melting point of about 56° C.), stearyl alcohol (having a melting point of about 58-59° C.), behenyl alcohol (having a melting point of about 71° C.), and mixtures thereof. These alcohols are known to have the above melting point. However, they often have lower melting points when supplied, since such supplied products are often mixtures of fatty alcohols having alkyl chain length distribution in which the main alkyl chain is a cetyl, stearyl or behenyl group. In the present invention, more preferred fatty alcohols are cetyl alcohol, stearyl alcohol and mixtures thereof.

Commercially available high melting point fatty alcohols useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan).

Together with a high melting point fatty alcohol, the compositions can further comprise a low melting fatty alcohol, for example, oleyl alcohol.

### Aqueous Carrier

The conditioning composition of the present invention comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 70% to about 90% water.

### Silicone Compound

The compositions of the present invention may, or may not, contain a silicone compound. It is believed that the silicone compound can provide smoothness and softness on dry hair. The silicone compounds herein can be used at levels by weight of the composition of preferably from about 0.1% to about 20%, more preferably from about 0.5% to about 10%, still more preferably from about 1% to about 8%.

Preferably, the silicone compounds have an average particle size of from about 1 micron to about 50 microns, in the composition.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

Preferred polyalkyl siloxanes include, for example, polydimethylsiloxane, polydiethylsiloxane, and polymethylphenylsiloxane. Polydimethylsiloxane, which is also known as dimethicone, is especially preferred. These silicone compounds are available, for example, from the General Electric Company in their Viscasil® and TSF 451 series, and from Dow Corning in their Dow Corning SH200 series.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

Silicone compounds useful herein also include amino substituted materials. Preferred aminosilicones include, for example, those which conform to the general formula (I):

(R₁)ₐG₃₋ₐ-Si—(—OSiG₂)ₙ-(—OSiG_{b}(R₁)_{2-b})ₘ—O-SiG₃₋ₐ(R₁)ₐ

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH3)₂ or —NH2, more preferably —NH2. Another highly preferred amino silicones are those corresponding to formula (I) wherein m=0, a=1, q=3, G=methyl, n is preferably from about 400 to about 600, more preferably about 500; and L is —N(CH3)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those having alkylamino substitutions as pendant groups of a silicone backbone. Highly preferred are those known as “amodimethicone”. Commercially available amodimethicones useful herein include, for example, BY16-872 available from Dow Corning. Some embodiments may include Silicone Quaternium-26.

The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; coloring agents, such as any of the FD&C or D&C dyes; perfumes; and sequestering agents, such as disodium ethylenediamine tetra-acetate; ultraviolet and infrared screening and absorbing agents such as benzophenones; and antidandruff agents such as zinc pyrithione.

### Low Melting Point Oil

The compositions may comprise one or more conditioning oils. Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils including pentaerythritol tetraisostearate, trimethylol ester oils, citrate ester oils, and glyceryl ester oils; poly α-olefin oils such as polydecenes; and mixtures thereof. Additional oils may include polyester oil or mono-, di, tri-ether or ester including triglycerides, such as caprylic capric triglyceride or vegetable oils such as coconut oil, soybean oil, rapeseed oil, cocoa butter, olive oil, palm oil, rice bran oil, and mixtures thereof.

In some embodiments, a conditioning oil may have a hydrophilic-lipophilic balance (HLB) of less than about 10. In some embodiments, the oil may be a mono, di, or tri ester or ether where the monomer units have a carbon chain of C2 to C16, preferably C4 to C10, or more preferably C6 to C8. In some embodiments, the oil may be a polyester with the hydrophobic monomer units (linear or branched) having carbon chains shorter than C16, preferably shorter than C12. Commercially available oil examples include, but are not limited to, Myritol 318 from BASF (caprylic/capric triglyceride), Plantasil Micro from BASF (dicaprylyl ether in emulsion form (Dicaprylyl Ether (and) Decyl Glucoside (and) Glyceryl Oleate)); or Citropol 1A from P2 science (Polycitronellol Acetate).

### Product Forms

The conditioning compositions of the present invention can be in the form of rinse-off products or leave-on products and can be formulated in a wide variety of product forms, including but not limited to pastes, creams, gels, emulsions, mousses, and sprays. The conditioning composition of the present invention is especially suitable for a rinse-off hair conditioner or for a no-rinse hair conditioner.

### Method of Use

The conditioning composition of the present invention is preferably used for a method of conditioning hair, the method comprising following steps:
(i) after shampooing hair, applying to the hair an effective amount of the conditioning composition for conditioning the hair; and
(ii) optionally, then rinsing the hair.

Effective amount herein is, for example, from about 0.1 ml to about 2 ml per 10 g of hair, preferably from about 0.2 ml to about 1.5 ml per 10 g of hair.

The conditioning composition of the present invention provides improved conditioning benefits, especially improved wet conditioning benefits after rinsing and improved dry conditioning, while maintaining wet conditioning benefit before rinsing. The conditioning composition of the present invention may also provide improved product appearance to consumer. Thus, a reduced dosage of the conditioning composition of the present invention may provide the same level of conditioning benefits as those of a full dosage of conventional conditioner compositions. Such reduced dosage herein is, for example, from about 0.3 ml to about 0.7 ml per 10 g of hair.

### Method of Manufacturing

The present invention is also directed to a method of manufacturing a hair conditioning composition as follows:

A method of making the hair conditioner composition comprising:
a. a basic amino acid;
b. a fatty acid;
c. a high melting point fatty alcohol; and
d. an aqueous carrier;
   said method comprising the following steps:
   a) Add water that is at a temperature higher than the temperature of the melting point of the fatty acid, the fatty alcohol, and mixture of them (about 80° C.-90° C.);
   b) Add basic amino acid in hot water (about 80° C.-90° C.) before adding of fatty alcohol and fatty acid;
   c) Prepare a homogeneous premix by combining a fatty acid and a fatty alcohol at a temperature that is higher than either of their individual melting points and add it into hot water (about 80° C.-90° C.;
   d) Cool the mixture below the phase transition temperature to form a gel network matrix.

### Method of Preparation

Water is prepared at from about 80° C. to about 90° C. Basic amino acid is added in the prepared hot water. Fatty alcohols and fatty acid then are added in the prepared hot solution and prepared dispersed homogenous mixture at about 85° C. After, the mixture is cooled down to below 40° C. with agitation. A lamellar gel network matrix with a Lβ phase is formed. If included, silicone compounds, perfumes, preservatives are added to the lamellar gel network matrix with a Lβ phase with agitation after the composition is cooled down to room temperature.

The present invention is also directed to a second method of manufacturing a hair conditioning composition as follows:
a) Add water that is at a temperature higher than the temperature of the melting point of the fatty acid, the fatty alcohol, and mixture of them;
b) Add fatty alcohols and wait until dissolved and dispersed homogenously;
c) Add fatty acid and wait until dissolved and dispersed homogenously;
d) Add basic amino acid and wait until dissolved and dispersed homogenously;
e) Cool the mixture below the phase transition temperature to form a gel network matrix; and
f) Add the remaining ingredients either before or after the gel network matrix formation, while maintaining pH of at least about 4.5.

An alternative method may be to prepare a homogeneous premix by combining a fatty acid and a fatty alcohol at a temperature that is higher than either of their individual melting points and add it into hot water (about 80-90° C.) instead of steps of b) and c). Another alternative method may be to add basic amino acid in hot water (about 80-90° C.) before adding of fatty alcohol and fatty acid.

A further alternative method of making a hair conditioner composition may comprise the following steps:

a) making a solid shape material by combining a basic amino acid, a fatty alcohol, and a fatty acid; and then b) combining a) in an aqueous chassis.

The method may further comprise the steps of adding additional ingredients such as silicone or oil compounds, perfumes, preservatives, esthetics if included, to the lamellar gel network matrix with a L_{β} phase. The inventive conditioning compositions of the present invention can be prepared by any conventional method well known in the art.

The pH of the finished product and of the composition during the making process after the cooling down step may be at least 4.5.

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

Table 1 below shows five formulations of hair conditioners. Examples 1 and 2 are inventive examples, comprising a basic amino acid, a fatty acid, a fatty alcohol, and aqueous carrier, which together form a lamellar gel network matrix with a L_{β} phase. The existence of the lamellar gel network matrix with a L_{β} phase is shown by the X-ray (SAX/WAX) analysis data. Comparative Example 3 is the same as Examples 1 and 2 except that Example 3 is absent any basic amino acid.

Small-angle x-ray scattering (“SAXS”) is used to resolve periodic structures in mesophases and is essentially an x-ray diffraction technique. It is used in conjunction with conventional wide-angle x-ray diffraction (“WXRD”) to characterize aggregate structures such as micelles, gel networks, lamella, hexagonal and cubic liquid crystals. The different mesophases that show periodic structures can be characterized by the relative positions (d-spacing) of their reflections as derived from the Bragg equation (d=λ/2 Sin θ) where d represents the interplanar spacing, λ the radiation wavelength and θ the scattering (diffraction) angle.

The one dimensional lamella gel network phase is characterized by the ratio of the interplanar spacings d₁/d₁, d₁/d₂, d₁/d₃, d₁/d₄, d₁/d₅ having the values 1:2:3:4:5 etc. in the SAXS region (long-range order) and one or two invariant reflection(s) in the WXRD region (short-range) centered around 3.5 and 4.5 Å over a broad halo background. Other mesophases (e.g. hexagonal or cubic) will have characteristically different d-spacing ratios.

Both WXRD and SAXS data are generated as described in more detail below in the Test Methods. As can be seen in Table 1, Inventive Examples 1 and 2 exhibit SAXS d-spacing and also WXRD reflection in the 3.5 to 4.5 range, indicating a lamellar gel network matrix. Example 3, in contrast, exhibits phase separation, which indicates that the lamellar gel network matrix with a L_{β} phase cannot form without arginine. Comparative Example 4 is also absent any basic amino acid and instead uses a conventional cationic surfactant. Qualitative analysis of the SAX peak intensity data shows that Inventive Examples 1 and 2 are able to more effectively form the lamellar gel network matrix with a L_{β} phase, compared with Example 4 using conventional cationic surfactant. Example 5 comprises a basic amino acid, arginine, and also a conventional cationic surfactant.

Inventive Example 1 and Comparative Example 4 were tested for their in-use experiences as rinse-off conditioners. The clean feel/squeaky feel on wet hair after rinsing was measured by a sensory Descriptive Analysis Panel (DAP) test using treated hair sample-tresses evaluated by eight highly trained expert panellists. Inventive Example 1 provided more squeaky feel after rinse as compared to Example 4, the formula with the conventional cationic surfactant. Another set of hair tresses were pre-treated with Examples 1 and 4 and dried overnight and evaluated for visual hair alignment and dry smooth look by the eight highly trained expert panellists. Again, Example 1 showed a higher score for hair alignment and dry smooth look than Example 4.

Inventive Example 1 and Comparative Example 5 were tested for their in-use experiences as leave-on hair conditioners. Example 1 provided less heavy coating or residue feel during application yet more frizz control as compared to Example 5. All test methods are disclosed in more detail below.

TABLE 1

| | | | Comparative | Comparative | Comparative |
|---|---|---|---|---|---|
| | Ex. 1 | Ex. 2 | Ex. 3 | Ex. 4 | Ex. 5 |
| Description | Inventive | Inventive | No basic | No basic | Basic amino |
| | Example 1 | Example 2 | amino acid; | amino acid; | acid plus a |
| | | | Stearic acid | Conventional | conventional |
| | | | | cationic | cationic |
| | | | | surfactant | surfactant |
| L-Arginine | 0.2 | 0.1 | — | — | 0.2 |
| Poly-lysine | — | 0.1 | — | — | — |
| Stearic Acid* | 1.95 | 1.95 | 1.95 | — | — |
| Stearamidopropyl | — | — | — | 1.95 | 1.95 |
| dimethylamine | | | | | |
| (SAPDMA) | | | | | |
| L-Glutamic Acid | — | — | — | 0.63 | 0.63 |
| Cetyl Alcohol | 1.68 | 1.68 | 1.68 | 1.68 | 1.68 |
| Stearyl Alcohol | 2.90 | 2.90 | 2.90 | 2.90 | 2.90 |
| Water | Q.S, | Q.S, | Q.S, | Q.S, | Q.S, |
| SAX: D-spacing (Å) | 540, 270, | 650, 320, | NA (phase | 570, 280, | — |
| | 180, | 210, | separation) | 190 | |
| WAX peak position | 4.10 | 4.10 | NA (phase | 4.10 | — |
| (Å) | | | separation) | | |
| SAX peak intensity | Stronger | Stronger | NA (phase | Weaker | — |
| | | | separation) | | |
| Squeaky feel after | 46 S | — | NA (phase | 30 | — |
| rinse (wet DAP ROC) | | | separation) | | |
| Alignment & Smooth | 73 S | — | NA (phase | 61 | — |
| (dry DAP ROC) | | | separation) | | |
| Heavy coating/ | 52 S | — | NA (phase | — | 73 |
| residue (wet DAP LOT) | | | separation) | | |
| Frizz | 34 S | — | NA (phase | — | 39 |
| (OSM LOT) | | | separation) | | |

*It has chain distribution of C16/C18 at around 50/50.
S = Significantly different

Tables 2 and 3 below contain more inventive examples. All inventive examples sufficiently form a lamellar gel network matrix with a L_{β} phase in formulation.

TABLE 2

| | Basic amino- acid (a) variation | | | Fatty acid (b) variation, | | |
|---|---|---|---|---|---|---|
| | Ex. 6 | Ex. 7 | Ex. 8 | Ex. 9 | Ex. 10 | Ex. 11 |
| Description | Arginine: | Arginine + | Histidine: | Arginine: | Arginine: | Arginine: |
| | Stearic | Histidine: | Stearic | Lauric | Behenic | Lauric |
| | Acid | Stearic | Acid | Acid | Acid | Acid |
| | | Acid | | (C-12) | (C-22) | (C-12) + |
| | | | | | | Behenic |
| | | | | | | Acid |
| | | | | | | (C-22) |
| L-Arginine | 0.22 | 0.11 | — | 0.22 | 0.22 | 0.22 |
| Histidine | — | 0.11 | 0.22 | — | — | — |
| Lauric Acid | — | — | — | 2.17 | — | 1.08 |
| Stearic Acid* | 2.17 | 2.17 | 2.17 | — | — | — |
| Behenic Acid | — | — | — | — | 2.17 | 1.08 |
| Oleic Acid | — | — | — | — | — | — |
| Cetyl | 1.75 | 1.75 | 1.75 | 1.75 | 1.75 | 1.75 |
| Alcohol | | | | | | |
| Stearyl | 3.02 | 3.02 | 3.02 | 3.02 | 3.02 | 3.02 |
| Alcohol | | | | | | |
| Water | Q.S, | Q.S, | Q.S. | Q.S, | Q.S, | Q.S, |
| Product | Stable | Stable | Stable | Stable | Stable | Stable |
| Stability | | | | | | |
| SAX: D- | 512, 256 | 530, 265, | 530, 265, | 392, 196 | 483, 246 | 530, 265 |
| spacing (Å) | | 174 | 174 | | | |
| WAX peak | 4.1 | 4.1 | 4.1 | 4.1 | 4.1 | 4.1 |
| position (Å) | | | | | | |
| Conditioning | Acceptable | Acceptable | Acceptable | Acceptable | Acceptable | Acceptable |

*It has chain distribution of C16/C18 at around 50/50.

TABLE 3

| | Fatty | | | | | Addition of conditioning oil | | |
|---|---|---|---|---|---|---|---|---|
| | Alcohol | | | | | or/and mix of | | |
| | variation | a:b ratio variation | | | | saturate/unsaturated fatty acids | | |
| | Ex. 12 | Ex. 13 | Ex. 14 | Ex. 15 | Ex. 16 | Ex. 17 | Ex. 18 | Ex. 19 |
| Description | Arginine: | Lower | Higher | Arginine: | Arginine: | Arginine: | Arginine: | Arginine: |
| | Stearic | Stable | Stable | Stearic | Lauric | Stearic | Stearic | Stearic |
| | Acid + | Arginine: Stearic | Arginine: | Acid | Acid | Acid + | Acid + | Acid + |
| | Cetyl | Acid | Stearic | (1:1) | (20:1) | Oil | Silicone | Oil + |
| | Alcohol | (1:40) | Acid | | | | | Silicone |
| | (C-16) | | (20:1) | | | | | |
| L-Arginine | 0.22 | 0.06 | 2.27 | 1.19 | 2.27 | 0.39 | 0.40 | 0.20 |
| Histidine | — | — | — | — | — | — | — | 0.20 |
| Lauric Acid | — | — | — | — | 0.11 | — | — | — |
| Stearic | 2.17 | 2.33 | 0.11 | 1.19 | — | 3.80 | 3.89 | 1.90 |
| Acid* | | | | | | | | |
| Behenic | — | — | — | — | — | — | — | 1.90 |
| Acid | | | | | | | | |
| Oleic Acid | — | — | — | — | — | — | 0.80 | — |
| Cetyl | 4.77 | 1.75 | 1.75 | 1.75 | 1.75 | 3.29 | 1.68 | 2.29 |
| Alcohol | | | | | | | | |
| Stearyl | — | 3.02 | 3.02 | 3.02 | 3.02 | 5.68 | 2.90 | 4.68 |
| Alcohol | | | | | | | | |
| Oleyl | — | — | — | — | — | — | — | 2.00 |
| Alcohol | | | | | | | | |
| Rapeseed | — | — | — | — | — | 2.00 | — | 2.00 |
| acid | | | | | | | | |
| Linoleic | — | — | — | — | — | — | 0.20 | — |
| acid | | | | | | | | |
| Caprylic | — | — | — | — | — | — | — | 1.00 |
| Capric | | | | | | | | |
| Triglyceride | | | | | | | | |
| Plantasil | — | — | — | — | — | 8.00 | — | 2.00 |
| Micro* | | | | | | | | |
| Dimethicone | — | — | — | — | — | — | — | 1.00 |
| Bis- | — | — | — | — | — | — | 1.00 | 0.25 |
| aminoprotpyl | | | | | | | | |
| dimethicone | | | | | | | | |
| Water | Q.S, | Q.S, | Q.S, | Q.S, | Q.S, | Q.S, | Q.S, | Q.S, |
| Product | Stable | Stable | Stable | Stable | Stable | Stable | Stable | Stable |
| Stability | | | | | | | | |
| SAX: D- | 540, 270, | 560, 280, | 331, | 209, 103 | 465,230 | 185,93, | 414, 206, | 298, 146, |
| spacing (Å) | 195 | 189 | 161 | | | 62 | 138 | 98 |
| WAX peak | 4.1 | 4.1 | 4.1 | 4.1 | 4.1 | 4.1 | 4.1 | 4.1 |
| position (Å) | | | | | | | | |
| Conditioning | Acceptable | Acceptable | Acceptable | Acceptable | Acceptable | Acceptable | Acceptable | Acceptable |

*Plantasil Micro from BASF (Dicaprylyl Ether (and) Decyl Glucoside (and) Glyceryl Oleate)
**It has chain distribution of C16/C18 at around 50/50.

While inventive conditioners may use fatty acids having anywhere from a C10 to C22 alkyl chain length, inventive conditioners using fatty acids having C12-C14 alkyl chains may improve the wet detangling of the hair conditioning composition, while maintaining a clean feel, as compared to inventive conditioners comprising C16-C22 alkyl chains. Table 4 shows five additional inventive examples. Examples 9, 20, 21, 6 and 10 have C12, C14, C16, C18, and C22 alkyl chains, respectively. Each was made and tested with a sensory panel for its ability to Remove Tangle during application, for Wet Slip during application, and for Dry Smooth Feel. As can be seen in Table 4, the shorter alkyl chain fatty acids (C12 and C14) of Inventive Examples 9 and 20 performed the best with the sensory panel. Panelists rated these attributes for Inventive Examples 9 and 20 as significantly higher than the same attributes for the control of Example 6 with a fatty acid alkyl chain of C18. The panelists rated these attributes for Examples 21 and 10 the same as for the control of Example 6.

TABLE 4

| | Ex. 9 | Ex. 20 | Ex. 21 | Ex. 6 | Ex. 10 |
|---|---|---|---|---|---|
| Description | C12 Lauric | C14 Myristic | Longer Carbon | Longer Carbon | Longer Carbon |
| | Acid | Acid | Chain | Chain | Chain |
| | | | C16 Palmitic | C18 Stearic | C22 Behenic |
| | | | Acid | Acid | Acid |
| L-Arginine | 0.22 | 0.22 | 0.22 | 0.22 | 0.22 |
| Lauric Acid | 2.17 | — | — | — | — |
| Myristic Acid | — | 2.17 | — | — | — |
| Palmitic Acid | — | — | 2.17 | — | — |
| Stearic Acid** | — | — | — | 2.17 | — |
| Behenic Acid | — | — | — | — | 2.17 |
| Cetyl Alcohol | 1.75 | 1.75 | 1.75 | 1.75 | 1.75 |
| Stearyl Alcohol | 3.02 | 3.02 | 3.02 | 3.02 | 3.02 |
| Water | Q.S, | Q.S, | Q.S, | Q.S, | Q.S, |
| Remove Tangle during | D | D | C | C | C |
| application * | | | | | |
| Wet Slip during | D | D | C | C | C |
| application * | | | | | |
| Description of Fast | Yes | Yes | Yes | Yes | Yes |
| Rinse/Clean Feel * | | | | | |
| Dry Smooth Feel * | D | C | B | C | C |
| Squeaky feel after rinse * | C | C | C | C | C |
| Alignment & Smooth * | C | C | C | C | C |

* Control is the Ex. 6 C18 Stearic Acid
**It has chain distribution of C16/C18 at around 50/50.
A = Obvious Significant lower intensity to attribute
B = Significant lower intensity to attribute
C = Equal intensity to attribute
D = Significant higher intensity to attribute
E = Obvious Significant higher intensity to attribute

In some embodiments, the ratio of the basic amino acid to the fatty acid, weight % to weight %, may be from about 20:1 to about 1:1, in some embodiments from about 20:1 to about 10:1. Table 5 below shows inventive examples with various ratios of basic amino acid to fatty acid (arginine to lauric acid). Examples 16 and 22, with the basic amino acid to fatty acid ratios of 20:1 and 10:1, respectively, performed the best with a sensory panel that evaluated the compositions for the following attributes: Remove Tangle during application, Dry Smooth Feel, Ease of Distribution to Bottom Wet Hair, and Ease of Distribution to Bottom Dry Hair. Other Examples evaluated had basic amino acid to fatty acid ratios of 40:1, 4:1, 2:1, and 1:1, all compared to the control (example 9), which had a ratio of 1:10.

TABLE 5

| | Ex. 16 | Ex. 22 | Ex. 23 | Ex. 24 | Ex. 25 | Ex. 26 |
|---|---|---|---|---|---|---|
| Description | Arginine: | Arginine: | Lower | Lower | Lower | Higher |
| | Lauric Acid | Lauric Acid | Arginine | Arginine | Arginine | Arginine |
| | ratio at 20:1 | ratio at 10:1 | Level: | Level: | Level: | Level: |
| | | | Lauric Acid | Lauric Acid | Lauric Acid | Lauric Acid |
| | | | ratio at 4:1 | ratio at 2:1 | ratio at 1:1 | ratio at 40:1 |
| L-Arginine | 2.27 | 2.17 | 1.91 | 1.51 | 1.19 | 2.33 |
| Lauric Acid | 0.11 | 0.22 | 0.43 | 0.80 | 1.19 | 0.06 |
| Cetyl Alcohol | 1.75 | 1.75 | 1.75 | 1.75 | 1.75 | 1.75 |
| Stearyl | 3.02 | 3.02 | 3.02 | 3.02 | 3.02 | 3.02 |
| Alcohol | | | | | | |
| Water | Q.S, | Q.S, | Q.S, | Q.S, | Q.S, | Q.S, |
| Remove | D | D | D | D | D | C |
| Tangle during | | | | | | |
| application * | | | | | | |
| Dry Smooth | D | D | C | C | C | C |
| Feel * | | | | | | |
| Squeaky feel | C | C | C | C | C | C |
| after rinse * | | | | | | |
| Alignment & | C | C | C | C | C | C |
| Smooth * | | | | | | |

Application feel for both rinse off and leave-on

| | | | | | | |
|---|---|---|---|---|---|---|
| Ease of | D | D | — | — | — | — |
| Distribution | | | | | | |
| to Bottom Wet | | | | | | |
| Hair * | | | | | | |
| Ease of | D | D | — | — | — | — |
| Distribution | | | | | | |
| to Bottom Dry | | | | | | |
| Hair * | | | | | | |

* Control is the Ex. 9 C12 Lauric Acid
A = Obvious Significant lower intensity to attribute
B = Significant lower intensity to attribute
C = Equal intensity to attribute
D = Significant higher intensity to attribute
E = Obvious Significant higher intensity to attribute

In some embodiments, the ratio of (a+b)/(a+b+c) (ratio of sum of basic amino acid and fatty acid to the sum of basic amino acid, fatty acid, and fatty alcohol, by weight) may be significant. A range from about 0.1 to about 0.9 may provide better detangling and also a more robust lamellar gel network matrix with a L_{β} phase structure. The range from 0.2 to 0.5 is even more effective. Table 6 shows the ratio for Examples 16, 27 and 28. In Example 16, the ratio is 0.33 and the composition shows an improved performance for Removing Tangles During Application and for Dry Smooth Feel, in comparison to Examples 27 and 28, which have ratios of 0.14 and 0.91, respectively.

TABLE 6

| | Ex. 16 | Ex. 27 | Ex. 28 |
|---|---|---|---|
| Description | (a + b)/ | (a + b)/ | (a + b)/ |
| | (a + b + c) = | (a + b + c) = | (a + b + c) = |
| | 0.33 | 0.14 | 0.91 |
| L-Arginine | 2.27 | 2.33 | 2.17 |
| Lauric Acid | 0.11 | 0.06 | 0.22 |
| Cetyl Alcohol | 1.75 | 1.75 | 1.75 |
| Stearyl Alcohol | 3.02 | 3.02 | 3.02 |
| Water | Q.S, | Q.S, | Q.S, |
| Remove Tangle during | D | B | B |
| application* | | | |
| Dry Smooth Feel* | D | B | C |
| Squeaky feel after rinse* | C | C | C |
| Alignment & Smooth* | C | C | C |

*Control is the Ex. 9 C12 Lauric Acid

In some embodiments, compositions comprising C12-C14 shorter fatty acid alkyl chains can provide improved wet and dry conditioning, in combination with high levels of arginine. In Table 7, Inventive Examples 16, 29, 30 and 31 all have a ratio of arginine to fatty acid of 20:1. When Examples 16, 29, 30 and 31 were compared to a control having an arginine to fatty acid ratio of 1:10, Examples 16 and 29, with the shorter chain fatty acids of C12 and C14, respectively, performed better than Examples 30 and 31, which had fatty acid chain lengths of C18 and C22, respectively. Examples 16 and 29 had significantly higher intensity for Dry Smooth Feel, Remove Tangle during application, and Alignment and Smooth.

TABLE 7

| | Ex. 16 | Ex. 29 | Ex. 30 | Ex. 31 |
|---|---|---|---|---|
| Description | Arginine:C12 | Arginine:C14 | Arginine:Longer | Arginine:Longer |
| | Lauric Acid ratio | Myristic Acid ratio | Carbon Chain C18 | Carbon Chain C22 |
| | at 20:1 | at 20:1 | Stearic Acid 20:1 | Behenic Acid 20:1 |
| L-Arginine | 2.27 | 2.27 | 2.27 | 2.27 |
| Lauric Acid | 0.11 | — | — | — |
| Myristic Acid | — | 0.11 | — | — |
| Stearic Acid** | — | — | 0.11 | — |
| Behenic Acid | — | — | — | 0.11 |
| Cetyl Alcohol | 1.75 | 1.75 | 1.75 | 1.75 |
| Stearyl Alcohol | 3.02 | 3.02 | 3.02 | 3.02 |
| Water | Q.S, | Q.S, | Q.S, | Q.S, |
| Dry Smooth Feel* | D | D | C | NA (phase |
| | | | | separation) |
| Remove Tangle | D | D | C | NA (phase |
| during application* | | | | separation) |
| Squeaky feel after rinse* | C | C | C | NA (phase |
| | | | | separation) |
| Alignment & Smooth | C | C | C | NA (phase |
| | | | | separation) |

*Control is the Ex. 9 C12 Lauric Acid
**It has chain distribution of C16/C18 at around 50/50.

In some embodiments, compositions comprising longer fatty acid chains, such as C22, may provide a coated dry conditioning feel without a greasy residue feel, especially for highly damaged hair. In Table 8, Example 37, a composition comprising a fatty acid with C22 performed better than Example 6, a composition comprising a fatty acid with C18, for providing a dry coated conditioning feel, according to the sensory panel.

TABLE 8

| | Ex. 6 | Ex. 37 |
|---|---|---|
| Chain length of fatty acid | C18 | C22 |
| | Arginine- | Arginine- |
| | StearicAcid | BehenicAcid |
| L-Arginine | 0.22 | 0.22 |
| Stearic Acid* | 2.17 | — |
| Behenic Acid | — | 2.17 |
| Cetyl Alcohol | 1.75 | 1.75 |
| Stearyl Alcohol | 3.02 | 3.02 |
| Water | Q.S, | Q.S, |
| SAX: D-spacing (Å) | 256, 168 | 483, 246 |
| WAX peak position (Å) | 4.1 | 4.1 |
| Dry Coated Conditioning Feel | Control | D |

**It has chain distribution of C16/C18 at around 50/50.
A = Obvious Significant lower intensity to attribute
B = Significant lower intensity to attribute
C = Equal intensity to attribute
D = Significant higher intensity to attribute
E = Obvious Significant higher intensity to attribute

### Test Methods

### 1. Wet Expert Sensory Method

This is expert sensory panel test method uses three highly expert sensory panel to evaluate specific attribute during wet stage hair treatment. The treatment protocol for the hair treatment is stated as follow:
a. Rinse 20 g of hair switches with water and squeeze water out from top to bottom once.
b. 1 ml conditioner was applied front and 1 ml conditioner was applied back.
c. Lather the product 30 strokes for 30 seconds on hair switch.
d. The hair was then rinse for 15 seconds front and 15 seconds back. and squeeze water out from top to bottom once.
   The sensory attribute evaluated during wet stage is mentioned below:
Wet Slip during application: Panelist will evaluate the finger speed from top to bottom of wet hair during application of 30 strokes on hair switch
Remove Tangle during application: Panellist will evaluate the finger detangling from top to bottom of wet hair after product application for three times
Squeaky Feel After Rinse: Panellist will evaluate the clean feel on wet hair after rinsing
   The score rating for the attribute is compared to the Control sample defined in each of test. Each category score is considered a meaningful difference from the other and shows that the attribute evaluated by all three highly sensitive panellist is consistent. A mix score data between the panellist, the score placed will be at lower score.
A=Obvious Significant lower intensity to attribute
B=Significant lower intensity to attribute
C=Equal intensity to attribute
D=Significant higher intensity to attribute
E=Obvious Significant higher intensity to attribute
Description Test for Fast Rinse/Clean Feel: Panellist will be asked to choose Yes or No when asked if the hair switch is fast to rinse and feels clean.
Ease of Distribution to Bottom Wet Hair: Panellist will evaluate the ease of spreading of product on Wet hair from root to tip during product application

### 2. Dry Expert Sensory Method

This is expert sensory panel test method uses three highly expert sensory panel to evaluate specific attribute during dry stage hair treatment. The treatment protocol for the hair treatment is stated as follow:
a. Rinse 20 g of hair switches with water and squeeze water out from top to bottom once.
b. 1 ml conditioner was applied front and 1 ml conditioner was applied back.
c. Lather the product 30 strokes for 30 seconds on hair switch.
d. The hair was then rinse for 15 seconds front and 15 seconds back. and squeeze water out from top to bottom once.
e. Leave overnight to dry
   The sensory attribute evaluated during dry stage is mentioned below:
Dry Smooth Feel: Panellist will evaluate the smooth surface feel of the hair
Alignment & Smooth: Panellist will evaluate the visual hair alignment and dry smooth look
Dry Coated Conditioning Feel: Panellist will evaluate the coated feel of the hair
Ease of Distribution to Bottom Dry Hair: Panellist will evaluate the ease of spreading of product on dry hair from root to tip for 30 lathering
   The score rating for the attribute is compared to the Control mentioned in each table. Each category score is considered a meaningful difference from the other and shows that the attribute evaluated by all three highly sensitive panellist is consistent. A mix score data between the panellist, the score placed will be at lower score.
A=Obvious Significant lower intensity to attribute
B=Significant lower intensity to attribute
C=Equal intensity to attribute
D=Significant higher intensity to attribute
E=Obvious Significant higher intensity to attribute

### 3. General Conditioning Sensory Method

This is expert sensory panel test method uses three highly expert sensory panel to evaluate specific attribute during dry stage hair treatment. The treatment protocol for the hair treatment is stated as follow:
a. Rinse 20 g of hair switches with water and squeeze water out from top to bottom once.
b. Apply 1 ml conditioner on front and apply 1 ml conditioner on back.
c. Lather the product 30 strokes for 30 seconds on hair switch.
d. Rinse for 15 seconds front and 15 seconds back. and squeeze water out from top to bottom once.
e. Leave overnight to dry

The sensory attribute evaluated during this method is notated as “Conditioning” in the Example tables. Panellist evaluated wet stage for wet spreadability and slip, and evaluated dry smoothness during dry stage, and was asked to find it either “Acceptable” or “Not Acceptable” (as conditioning feel).

### 4. DSC Analysis Method

The differential scanning calorimetry (DSC) is a convenient tool as it gives the phase transition (melting or freezing) temperature of formed structure, as well as the thermal energy of melting and freezing. The procedure method comprising the following steps:
1. Equilibrate 0.00° C.
2. Ramp 5.00° C./min to 90.00° C.
3. Isothermal 5.0 min
4. Ramp 5.00° C./min to 0.00° C.
5. Isothermal 5.0 min

The description test for DSC Peak >30° C.: The DSC graph at Ramp 5.00° C./min to 90.00° C. will gives the melting phase transition temperature of formed structure. If a peak is observed higher than 30° C., then it will be stated as Yes.

### 5. Product Stability Visual Assessment

Stability is the visual assessment to ensure the product is consistently stable over a specific period. The composition of product was placed in three different condition—5° C., 25° C. and 40° C. The assessment will be taken at different interval:
1) Stable as made
2) Stable after one week

The description test for Product Stability: If the visual assessment of the product is stable at different interval, it will be stated as Stable. If the visual assessment of the product exhibit phase separation at either of the interval, it will be stated as Phase Separation.

### 6. SAX/WAXS Analysis Method

Small-angle x-ray scattering (“SAXS”) as used to resolve periodic structures in mesophases is essentially an x-ray diffraction technique. It is used in conjunction with conventional wide-angle x-ray diffraction (“WXRD”) to characterize aggregate structures such as micelles, gel networks, lamella, hexagonal and cubic liquid crystals. The different mesophases that show periodic structures can be characterized by the relative positions (d-spacing) of their reflections as derived from the Bragg equation (d=λ/2 Sin θ) where d represents the interplanar spacing, λ the radiation wavelength and θ the scattering (diffraction) angle.

The one dimensional lamella gel network phase is characterized by the ratio of the interplanar spacings d₁/d₁, d₁/d₂, d₁/d₃, d₁/d₄, d₁/d₅ having the values 1:2:3:4:5 etc. in the SAXS region (long-range order) and one or two invariant reflection(s) in the WXRD region (short-range) centered around 3.5 and 4.5 Å over a broad halo background. Other mesophases (e.g. hexagonal or cubic) will have characteristically different d-spacing ratios.

Both WXRD and SAXS data are collected simultaneously in high resolution collimation for 90 minutes on a Xenocs Xeuss 2.0 with Dectris Pilatus 100K detector (WXRD) and Dectris Pilatus 3R 200K-A detector (SAXS) at a sample to detector distance of 163.13 mm (WXRD) and 2492.37 mm (SAXS). The setup has an evacuated chamber. The specimen is injected into a quartz-glass capillary tube (diameter=2.0 mm, length=80 mm, wall thickness=0.01 mm), mounted in grooved capillary sample holder and placed in the path of the x-ray beam. Data are collected and analyzed using the Xenocs Foxtrot software. The 2-D data are azimuthally integrated and reduced to intensity versus scattering vector (q) or its d equivalent by the SAXS utilities software.

### 7. Rheology Method

Rheology is used to evaluate and characterise product samples. The two key rheology methods identified are mentioned below:
Shear Stress at 950s⁻¹ via flow curve: This is the method to ramp up shear rate logarithmically from 0.1 to 1000 s⁻¹ in 1 min using a cone & plate geometry, and to read the shear stress value a (Pa) at shear rate 950 s⁻¹.
Oscillatory Measurement G′/G″: This is the Oscillatory stress method where it ramps from 0.1 to 100 Pa to measure storage modulus, G′ and loss modulus, G″ to give viscoelasticity information of “resting” state of sample, and the yield stress value a (Pa), which is the stress required to permanently deform (starts to flow).
   The acceptable rheology range for shear stress is from 5 Pa until 1500 Pa. As for oscillatory measurement, the range for storage modulus, G′ is from 30 Pa until 45000 Pa, and loss modulus, G″ is from 10 Pa until 20000 Pa.

### Wet and Dry DAP Sensory for Rinse-Off

Squeaky Feel after Rinse (Wet DAP ROC)

Clean feel on wet hair after rinsing is evaluated by squeaky feel measured by sensory DAP-Descriptive Analysis Panel test using treated hair sample-tresses evaluated by eight highly trained expert panellists. 0.4 ml of the composition is applied to 4 g of a hair sample. After spreading the composition on the hair sample, it is rinsed with water for about 15 seconds. Water is squeezed from the hair sample, then the hair sample is evaluated for tactile feel on a scale from 1 to 100, with a higher number being a more squeaky feel after rinse. A difference of 5 or more points is considered a meaningful difference.

### Alignment & Smooth (Dry DAP ROC)

Another set of hair tresses are pre-treated with the composition and dried overnight, then evaluated for visual hair alignment and dry smooth look by eight highly trained expert panellists. The scale is from 1 to 100, with a higher number representing greater alignment and smoothness. A difference of 5 or more points is considered a meaningful difference.

### Heavy Coating/Residue (Wet DAP Sensory for Leave-on)

Clean feel during application as a leave-on treatment is evaluated by a heavy coating/residue rating as measured by a sensory DAP-Descriptive Analysis Panel test using treated hair sample-tresses as evaluated by eight highly trained expert panellists. 0.4 ml of the composition is applied to 4 g of a hair sample. After spreading the composition on the hair sample, the heavy coating/residue was evaluated on a scale from 1 to 100, with a higher number representing a more heavy coating or residue feel. A difference of 5 or more points is considered a meaningful difference.

### OSM (Optical Sectioning Method) Frizz Measure for Leave-on

A set of tresses are treated with hair care products and then exposed to high humidity conditions. The tresses are then hung vertically, and their shape measured by a laser scanning system. The volume of the tress and the fibres at the outer surface associated with frizzy hair are calculated with lower values indicating smaller volume and lower amounts of frizz. The values corresponding to volume and frizz of the sets of tresses corresponding to different hair treatments are tested for statistically significant differences at 95% confidence intervals.

### Examples/Combinations

A. A hair conditioner composition comprising:
   a) a basic amino acid;
   b) a fatty acid;
   c) a high melting point fatty alcohol; and
   d) an aqueous carrier;
      wherein a) through d) form a lamellar gel network matrix with a L_{β} phase.
B. The composition of paragraph A, wherein the mixture of a, b, and d have a pH of at least about 4.5.
C. The composition of any one of paragraphs A or B, wherein the hair conditioner composition comprises from about 0.01% to about 15% of the basic amino acid, by weight of the hair conditioner composition.
D. The composition of any one of paragraphs A to C, wherein the hair conditioner composition comprises from about 0.01% to about 15% of fatty acids, by weight of the hair conditioner composition.
E. The composition of any one of paragraphs A to D, wherein the hair conditioner composition comprises at least about 60% of an aqueous carrier, by weight of said hair conditioner composition.
F. The composition of any one of paragraphs A to E, wherein the basic amino acid comprises more than two amine groups.
G. The composition of any one of paragraphs A to F, wherein the ratio of (a+b) to (a+b+c) is about 0.1 to about 0.9.
H. The composition of any one of paragraphs A to G, wherein the ratio of a to b is from about 1:40 to about 40:1.
I. The composition of any one of paragraphs A to H, wherein the fatty acid comprises saturated and unsaturated fatty acids.
J. The composition of paragraph I, wherein the ratio of saturated fatty acids to unsaturated fatty acids is from about 8:1 to about 1:4.
K. The composition of any one of paragraphs A to J, wherein the fatty acid comprises from about 0.3% to about 15% of unsaturated fatty acid, by weight of the hair conditioner composition.
L. The composition of any one of paragraphs A to K, wherein the composition further comprises conditioning oils.
M. The composition of paragraph L, wherein the conditioning oil is a non-silicone.
N. The composition of any one of paragraphs L or M, wherein the conditioning oils are in preformed emulsion form, with a particle size at most about 500 nm.
O. The composition of any one of paragraphs L to N, wherein the conditioning oils have an HLB of less than about 10.
P. The composition of any one of paragraphs A to O, further comprising additional amino acids that when all amino acids are combined the iso-electrostatic point is higher than 7.
Q. The composition of any one of paragraphs A to P, wherein the basic amino acid is selected from the group consisting of arginine, lysine, histidine, and combinations thereof.
R. The composition of any one of paragraphs A to Q, wherein the fatty acid has C10-C22 alkyl chains.
S. The composition of any one of paragraphs A to R, wherein the ratio of (a+b) to (a+b+c) is from about 0.2 to about 0.5.

T. A method of making a hair conditioner composition, comprising the following steps:

a) heat water to between 80° C. and 90° C.;
b) add a basic amino acid into the heated water;
c) Prepare a homogeneous premix by combining a fatty acid and a fatty alcohol at a temperature that is higher than either of their individual melting points and add the premix into the heated water;
d) Cool the mixture of a), b), and c) to below its phase transition temperature to form a gel network matrix.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioner composition comprising:
a) a basic amino acid;
b) a fatty acid;
c) a high melting point fatty alcohol; and
d) an aqueous carrier;
wherein a) through d) form a lamellar gel network matrix with a L_{β} phase.

2. The hair conditioner composition of claim 1, wherein the mixture of a, b, and d have a pH of at least about 4.5.

3. The hair conditioner composition of claim 1, wherein the hair conditioner composition comprises from about 0.01% to about 15% of the basic amino acid, by weight of the hair conditioner composition.

4. The hair conditioner composition of claim 1, wherein the hair conditioner composition comprises from about 0.01% to about 15% of fatty acids, by weight of the hair conditioner composition.

5. The hair conditioner composition of claim 1, wherein the hair conditioner composition comprises at least about 60% of an aqueous carrier, by weight of said hair conditioner composition.

6. The hair conditioner composition of claim 1, wherein the basic amino acid comprises more than two amine groups.

7. The hair conditioner composition of claim 1, wherein the ratio of (a+b) to (a+b+c) is about 0.1 to about 0.9.

8. The hair conditioner composition of claim 1, wherein the ratio of a to b is from about 1:40 to about 40:1.

9. The hair conditioner composition of claim 1, wherein the fatty acid comprises saturated and unsaturated fatty acids.

10. The hair conditioner composition of claim 9, wherein the ratio of saturated fatty acids to unsaturated fatty acids is from about 8:1 to about 1:4.

11. The hair conditioner composition of claim 1, wherein the fatty acid comprises from about 0.3% to about 15% of unsaturated fatty acid, by weight of the hair conditioner composition.

12. The hair conditioner composition of claim 1, wherein the composition further comprises a conditioning oil.

13. The hair conditioner composition of claim 12, wherein the conditioning oil is a non-silicone.

14. The hair conditioner composition of claim 12, wherein the conditioning oil is in preformed emulsion form, with a particle size at most about 500 nm.

15. The hair conditioner composition of claim 13, wherein the conditioning oil has an HLB of less than about 10.

16. The hair conditioner composition of claim 1, further comprising additional amino acids that when all amino acids are combined the iso-electrostatic point is higher than 7.

17. The hair conditioner composition of claim 1, wherein the basic amino-acid is selected from the group consisting of arginine, lysine, histidine, and combinations thereof.

18. The hair conditioner composition of claim 1, wherein the fatty acid has C10-C22 alkyl chains.

19. The hair conditioner composition of claim 1, wherein the ratio of (a+b) to (a+b+c) is from about 0.2 to about 0.5.

20. A method of making a hair conditioner composition, comprising the following steps:
a) heat water to between 80° C. and 90° C.;
b) add a basic amino acid into the heated water;
c) Prepare a homogeneous premix by combining a fatty acid and a fatty alcohol at a temperature that is higher than either of their individual melting points and add the premix into the heated water;
d) Cool the mixture of a), b), and c) to below its phase transition temperature to form a gel network matrix.
